(19) United States
(12) Patent Application Publication
(10) Pub. No.: US 2020/0237944 A1
(43) Pub. Date: Jul. 30, 2020

(54) Systems and Methods for Sterilization

(71) Applicants: Patrick Leamy, Flemington, NJ (US); Qing-Qing Qiu, Branchburg, NJ (US); Michael S. Pohle, Flemington, NJ (US); Jason Michael Pomerleau, Somerville, NJ (US); Jerome Connor, Westminster, CO (US)

(72) Inventors: Patrick Leamy, Flemington, NJ (US); Qing-Qing Qiu, Branchburg, NJ (US); Michael S. Pohle, Flemington, NJ (US); Jason Michael Pomerleau, Somerville, NJ (US); Jerome Connor, Westminster, CO (US)

(73) Assignee: LifeCell Corporation, Madison, NJ (US)

(21) Appl. No.: 16/840,041

(22) Filed: Apr. 3, 2020

Related U.S. Application Data
(63) Continuation of application No. 15/185,280, filed on Jun. 17, 2016.
Application No. 15/185,280 is a continuation of application No. 14/206,793, filed on Mar. 12, 2014, now abandoned.
Application No. 14/206,793 is a continuation of application No. 12/939,669, filed on Nov. 4, 2010, now abandoned.
(60) Provisional application No. 61/258,490, filed on Nov. 5, 2009.

(51) Int. Cl. A61L 2/26; A61L 2/00; A61L 2/20; A61B 50/30

(52) U.S. Cl. CPC A61L 2/26; A61L 2/0082; A61L 2/206; A61L 2202/181; A61L 2202/24; A61B 2050/3014; A61B 2050/316; A61B 50/30

(57) ABSTRACT

Systems for sterilization of tissues, including acellular tissue matrices, comprising a package having a portion permeable to supercritical carbon dioxide and a portion impermeable to moisture are described. Methods of sterilizing acellular tissue matrices from soft tissues or demineralized bone are provided.

This application is a continuation application of U.S. Utility application Ser. No. 15/185,280, filed on Jun. 17, 2016, which is a continuation application of U.S. Utility application Ser. No. 14/206,793, filed on Mar. 12, 2014, which is a continuation of U.S. Utility application Ser. No. 12/939,669, filed on Nov. 4, 2010, which claims the benefit of U.S. Provisional Application No. 61/258,490, filed on Nov. 5, 2009, all of which are incorporated herein by reference in their entirety.

The present disclosure relates to the field of tissue sterilization.

Present methods for sterilization of tissues include gamma-irradiation, e-beam, and ethylene oxide (EO). Among them, gamma-irradiation and e-beam are known to alter the structure and characteristics of biomaterials through crosslinking and/or degradation of the collagen matrix. EO sterilization typically operates at temperatures around 60° C., which is above the melting temperature of collagen matrix in biological materials, such as, for example, dermal tissues. EO is also a recognized carcinogen, and residual EO in biological materials can cause hemolysis and other toxic reactions. Further, gamma-irradiation and e-beam can eliminate or significantly reduce osteoinductivity of demineralized bone matrix.

Accordingly, there is a need for improved systems and methods for sterilization of tissues, including acellular tissue matrices.

This discussion of the background disclosure is included to place the present disclosure in context. It is not an admission that any of the background material previously described was published, known, or part of the common general knowledge at the priority date of the present disclosure and claims.

According to certain embodiments, a packaging system for a medical device is disclosed. The system comprises a first portion, that is permeable to supercritical carbon dioxide (SC—CO₂) and a sterilant, and a second portion, that is impermeable to moisture.

According to certain embodiments, a method of terminal sterilization is disclosed, comprising treating a medical device in a supercritical carbon dioxide (SC—CO₂) chamber with a sterilant, wherein the device is packaged in an outer package prior to treating the device in the SC—CO₂ chamber, wherein the outer package comprises a first and a second portion, and wherein the first portion is permeable to the sterilant and the second portion is impermeable to moisture.

According to certain embodiments, a packaged tissue product is disclosed. The product comprises an acellular matrix, wherein the matrix has been treated in a SC—CO₂ chamber with a sterilant, wherein the matrix is packaged in a outer package prior to treating the matrix in the SC—CO₂ chamber, wherein the outer package comprises a first portion and a second portion, and wherein the first portion is permeable to the sterilant and the second portion is impermeable to moisture.

According to certain embodiments, a method of sterilization is provided. The method can comprise selecting a medical device for sterilization; placing the device in a first outer package, wherein the outer package comprises a first portion that is permeable to SC—CO₂ and a sterilant and second portion that is impermeable to moisture; and treating the device with SC—CO₂ and the sterilant.

According to certain embodiments, a packaged medical device is provided. The device can include a sealed outer package that is impermeable to moisture; an inner package that is permeable to SC—CO₂ and a sterilant; and a medical device contained within the inner package.

### BRIEF DESCRIPTION OF THE DRAWINGS

FIG. 1 is perspective view of one exemplary embodiment of a packaging system.

FIG. 2 is a phase diagram of CO₂.

FIG. 3 is a diagram of process pressure vs. time flow, according to certain embodiments.

FIG. 4 is a log-kill plot of model resistant bioburden, according to certain embodiments.

FIG. 5 is a log-kill plot of model resistant bioburden, according to additional embodiments.

FIG. 6 is a plot summarizing the effects of certain embodiments on tissue weight by enzyme digestion analysis.

### DESCRIPTION OF EXEMPLARY EMBODIMENTS

In this application, the use of the singular includes the plural unless specifically stated otherwise. In this application, the use of “or” means “and/or” unless stated otherwise. Furthermore, the use of the term “including,” as well as other forms, such as “includes” and “included,” is not limiting. Also, terms such as “element” or “component” encompass both elements and components comprising one unit and elements and components that comprise more than one subunit, unless specifically stated otherwise. Also, the use of the term “portion” may include part of a moiety or the entire moiety.

All documents, or portions of documents, cited in this application, including but not limited to patents, patent applications, articles, books, and treatises, are hereby expressly incorporated by reference in their entirety for any purpose.

The term “sterilization,” as used herein, generally refers to the inactivation or elimination of viable microorganisms.

The term “bioburden,” as used herein, generally refers to the number of contaminating microbes on a certain amount of material.

The term “tissue” will be understood to refer to intact tissue or components of tissues, including acellular tissue matrices.

The present disclosure relates to systems and methods for sterilization of medical devices. Some exemplary embodiments relate to sterilization using supercritical carbon dioxide (SC—CO₂). Supercritical carbon dioxide sterilization involves the use of SC—CO₂, alone or with the addition of one or more sterilants, for bioburden reduction. Supercritical carbon dioxide has unique properties that make it an appealing medium for sterilization. Its high diffusion characteristics allow for deep penetration into materials. In addition, it is nontoxic and can be easily removed by depressurization and out-gassing. Further, SC—CO₂ can be effective at inactivating a variety of microorganisms.

In some exemplary embodiments, SC—CO₂ may be used to sterilize a biocompatible material. In some embodiments, the biocompatible material may be a material that facilitates revascularization and cell repopulation. For example, in certain embodiments, the material can include an acellular tissue matrix (ATM). Additionally, in some embodiments, the biocompatible material may be demineralized bone matrix (DBM), such as, for example ALLOCRAFT™DBM, Lifecell Corporation (Branchburg, N.J.). In certain embodiments, the DBM bone is osteroinductive after sterilization.

Some exemplary embodiments may include treating a medical device with a combination of SC—CO₂ and a sterilant to further enhance the inactivation of microbes. Such sterilants may include, for example, peracetic acid (PAA), which can be bactericidal, fungicidal, virucidal, and sporicidal. Use of a sterilant in conjunction with SC—CO₂ in various embodiments disclosed herein, may facilitate achieving industrial level sterilization with a Sterility Assurance Level (SAL) of 10⁻⁶ (i.e., a probability of 1 in 1,000,000 of finding a non sterile device). In some embodiments, combining SC—CO₂ with a sterilant may facilitate achieving industrial sterilization of ATM without causing significant changes in susceptibility to collagenase digestion and in mechanical properties (e.g., tear strength, tensile strength) of the ATM. In other embodiments, combining SC—CO₂ with a sterilant may facilitate achieving industrial sterilization of DBM without affecting the osteoinductivity of the DBM.

Some exemplary embodiments of the present disclosure can be used for terminal sterilization of medical devices or tissues to provide a sterile device in a sealed package, thus avoiding the subsequent microbial contamination that may occur if a device is packaged or transferred after sterilization.

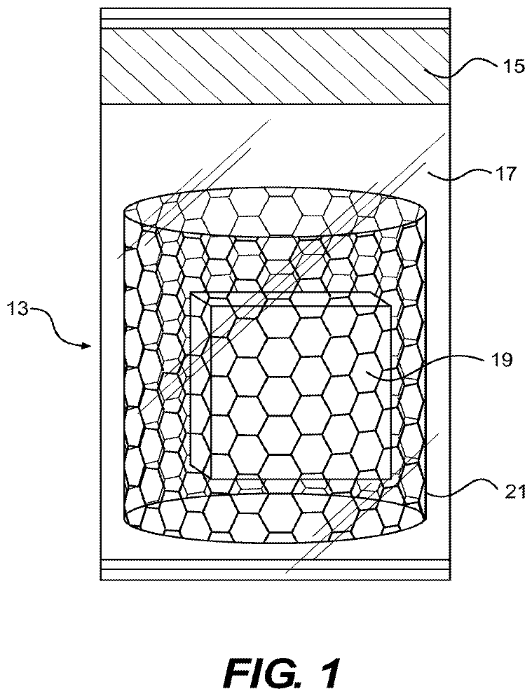

FIG. 1 provides a perspective view of one exemplary embodiment of a packaging system, according to certain embodiments. The packaging system may comprise an outer package 13. The outer package 13 may comprise a first portion 15 that is permeable to SC—CO₂ and a sterilant. First portion 15 may be impermeable to bacteria and may function as a sterile barrier. Suitable materials that can function as a sterile barrier and is permeable to SC—CO₂ and a sterilant may include paper or flashspun high-density polyethylene fibers, such as, for example, TYVEK®, DuPont Company (Wilmington, Del.). In some embodiments, the permeable portion may comprise medical grade paper. The outer package 13 may further comprise a second portion 17 that is impermeable to moisture. Second portion 17 may comprise, for example, foil. Second portion 17 may allow for the medical device to be sealed within a moisture tight enclosure subsequent to sterilization without transferring the device to a separate container. This will facilitate sterilization of hydrated tissues and/or other water containing devices, which need to be stored in moisture-tight enclosures. The storage solution of the enclosures should not interfere with SC—CO₂-PAA sterilization, i.e., by containing free radical scavengers.

In some embodiments, the packaging system may further comprise an inner package 19. Inner package 19 may be permeable to SC—CO₂ and a sterilant, and may comprise, for example, TYVEK®. Inner package 19 may be configured to contain the medical device and to be enclosed by outer package 13.

In some embodiments, the packaging system may also include an inner structure 21 to hold the second portion 17 open. Inner structure 21 may be permeable to SC—CO₂ and a sterilant and may comprise various shapes and sizes sufficient to maintain an opening in second portion 17, depending on the configuration of second portion 17. For example, as depicted in FIG. 1, inner structure 21 may comprise a mesh configured to surround the medical device and, if present, inner package 19. This mesh can include a cylindrical or tube shape. Inner structure 21 may be permeable to SC—CO₂ and a sterilant. In some embodiments, second portion 17 may be impermeable to SC—CO₂ and a sterilant. In such embodiments, the opening maintained in second portion 17 by inner structure 21 may allow for exposure of the medical device to SC—CO₂ and the sterilant.

In some embodiments, the medical device may first be packaged in inner package 19. Inner package 19 may then be positioned within inner structure 21, and inner structure 21 may, in turn, be placed within second portion 17 of outer package 13. Subsequently, first portion 15 of outer package 13 may then be sealed. The seal is made just below portion 15. The medical device may then be placed in a supercritical carbon dioxide chamber and treated with SC—CO₂ and a sterilant. Finally, after being treated with SC—CO₂ and a sterilant, second portion 17 of outer package 13 may then be sealed. The TYVEK® header may be removed after sterilization.

Exemplary Sterilization Process

In certain embodiments, super-critical carbon dioxide (SC—CO₂) can serve as an inert carrier for the delivery of sterilants. Is some embodiments, the sterilants can include, peracetic acid (PAA). In various embodiments, the sterilant can include PAA and hydrogen peroxide (H₂O₂). SC—CO₂ exhibits properties of both the gaseous and liquid physical states. It has the viscosity of a liquid and the transport efficiency of a gas which allow for efficient delivery with high penetration properties.

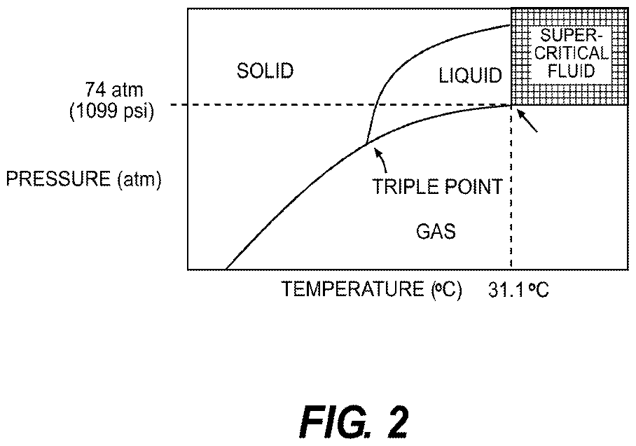

FIG. 2 displays a phase diagram for the conversion of CO₂ to the super-critical state. The super-critical state is a unique physical state that is achieved at a specific temperature and pressure combination termed the “critical point”. The super-critical state is absolute once the critical point is reached within the environment and the pressure and temperature are uniform throughout a super-critical environment.

In certain embodiments during the sterilization process, CO₂ is pumped into the chamber and the pressure and temperature are modulated until the critical point is surpassed to produce a super-critical state within the chamber. The pressure and temperature are monitored to maintain the required super-critical state pressure/temperature values for the duration of the processing run. If either the pressure or temperature range falls out of the required range, the run is registered as a failure. The real-time measurements of the temperature and pressure values are recorded and can be produced as a hard-copy printout.

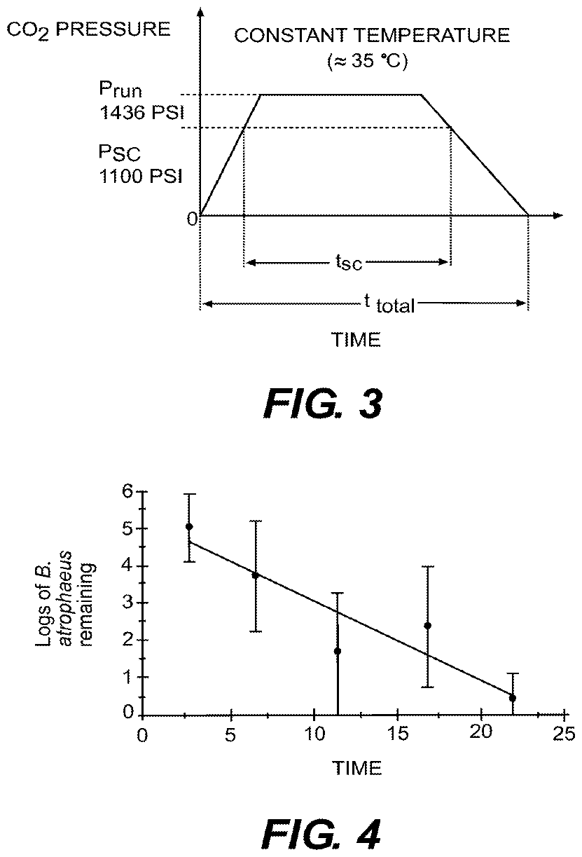

FIG. 3 is a diagram of process pressure vs. time flow, according to certain embodiments. During the sterilization process, an instrument provides heat to a treatment chamber to maintain a constant temperature of about 35° C., while the pressure is increased to above the critical point. Once the preset super-critical state (P=1346 psi, T=35° C.) is achieved, the sterilization time is initiated. In various embodiments, during the validated sterilization process, under super-critical conditions, both the pressure and temperature are constant. In some embodiments, the only variable for the sterilization process is the exposure time.

As FIG. 3 illustrates, there is a pre-sterilization time period during which the critical pressure is achieved. After that time, the pressure and temperature are held constant at values within the super-critical phase requirements. The sterilization time is then initiated, and the required exposure time is executed (t_{sc}). Following the completion of the sterilization exposure phase, the chamber pressure is reversed to allow retrieval of the sterile samples. The complete process of achieving the super-critical phase and its reversal are represented by the total process time (tₜₒₜₐₗ).

Sterilant Components

The sterilant component of the sterilization system consists of a stock solution that contains PAA and H₂O₂ (Sigma Cat No #269336), which is diluted with sterile distilled water at the time of use. In some embodiments, the PAA and H₂O₂ have concentrations in the sterilant of 12%±2.0% and 2.0%±1.0%, respectively. Thus, the concentrations of PAA and H₂O₂ inside the chamber during a sterilization process would be approximately 54 ppm and 9 ppm, respectively.

At the onset of the sterilization process, the sterilant is placed into the SC—CO₂ chamber. Due to the pressure chamber configuration, the PAA/H₂O₂ concentration cannot be monitored during the sterilization process, but the presence of PAA/H₂O₂ throughout the chamber can be confirmed by PAA and H₂O₂ test strips placed at different locations of the chamber during the IQ/OQ validation of the equipment. In various embodiments, the process can achieve SAL=10⁻⁶ sterilization.

In certain embodiments, the sterilization process is used to sterilize materials with the packaging system described above.

### Example 1: Sterilization

The first step of this method is to identify the natural bioburden of the product undergoing sterilization. Acellular porcine dermis was produced using LifeCell's porcine tissue processing, and samples were obtained prior to sterilization from 17 production lots over 3 weeks. A suitable process for preparing acellular tissue matrix is described in Xu et al., Tissue Engineering Part A. July 2009, 15(7): 1807-1819, but any suitable acellular tissue matrix can be sterilized with the disclosed process. The bioburden data was collected, expanded, and identified by Biotest Labs (Minneapolis, Minn.) and the results are provided in Table 1.

TABLE 1
Native Bioburden of Tissue Prior to Sterilization

| Microorganism | Classification |
|---|---|
| Enterobacter aerogenes | gram negative |
| Staphylococcus cohnii | gram positive |
| Staphylococcus haemolyticus | gram positive |
| Staphylococcus species | gram positive |
| Debaryomyces hansenii | yeast |

The second step of this method is to identify which microorganisms within the samples have the most resistance to the sterilization process. In addition to the established bioburden, a resistant model microorganism is included during this testing phase. Bacillus atrophaeus (spore form) was chosen due to its known high resistance to chemical sterilization, including PAA. These organisms were tested for resistance to the process by two methods. First, each organism was grown to high titer in solution and treated as a suspension. Secondly, tissue was inoculated individually with each organism, which were allowed to grow on the tissue until stationary growth was achieved. Both arms were treated with the sterilization, and the log of remaining bioburden was determined. For both the liquid suspension and tissue treatment, sterilization time was one minute wherein the concentrations of PAA and H₂O₂ inside the sterilant ranged from 10-14% and 1-3%, respectively. Table 2 displays the results of this testing following a short exposure to SC—CO₂ with sterilants.

TABLE 2
Determination of the Most Resistant Microorganism (log reduction)

| Microorganism | Liquid Suspension | Tissue |
|---|---|---|
| E. aerogenes | >6.5 logs | 7.0 logs |
| S. cohnli | >6.6 logs | >10.1 logs |
| S. haemolyficus | N/A | >10.1 logs |
| S. species | >6.7 logs | >10.1 logs |
| D. hansenii | >6.0 logs | 8.4 logs |
| B. atrophaeus | 1.6 logs | 4.6 logs |

This data clearly identified the model organism (B. atrophaeus spores) as the most resistant organism to the sterilization process and thus, it was used as the representative organism for the final phase of the validation.

The final step of the validation method is to determine the linearity and the D₁₀ value for the sterilization process using the most resistant microorganism. D₁₀ is the time required to achieve a 90% reduction in the active bacteria population.

For the final phase, tissue samples at the final step in the process were inoculated with 10₈ logs of B. atrophaeus spores and packaged in the final package configuration. The samples were placed in the sterilization apparatus in a fixed orientation, and the sterilant (minimum specification concentration) was added to the chamber. The process was run under constant pressure and temperature for increasing super-critical exposure times. Ten samples were tested at each time point. The tissue samples were extracted and enumerated to determine the remaining logs of the reporter organism. FIG. 4 displays the results of the validation study.

This validation data set demonstrates that the sterilization process produces a linear sterilization profile over time.

The following formula can be applied to determine the required dose (i.e., super-critical exposure time) to achieve an SAL=10⁻⁶ for the sterilization process with acellular porcine dermis.

For the sterilization:

the exposure time=D₁₀×[6+log(100+bioburden)]

To determine the endogenous bioburden of the product, 10 samples from 3 lots (based on ISO 11737-1) at a SIP=1 were produced, and the bioburden was enumerated prior to the terminal sterilization process. The resulting bioburden value was determined to be 1.6 cfu. 100 cfu was conservatively chosen as the endogenous bioburden of the product. Applying 100 cfu to “bioburden” in formula 1 yields the following outcome:

$$\begin{matrix}Super \text{-} critical time = 4.6 \times [ 6 + log ( 200 ) ] \\ = 4.6 \times [ 8.3 ] \\ = 38.2 minutes\end{matrix}$$

This is the SC—CO₂ treatment time that will yield an SAL=10⁻⁶ for the acellular porcine dermal product.

### Example 2: Use of Various Package Configurations to Facilitate Supercritical Carbon Dioxide Sterilization of Porcine Tissue Matrix

In a basic configuration, porcine tissue matrix is packaged in a TYVEK® pouch, which is sealed prior to supercritical carbon dioxide sterilization. In another configuration, the tissue is packaged within a TYVEK® pouch, which is placed in a foil pouch with a TYVEK® header. This “header pouch” is sealed along the TYVEK® Header/foil interface prior to supercritical carbon dioxide sterilization. After sterilization, the pouch is sealed at the foil-foil interface to yield a barrier to microorganisms and to moisture.

Various configurations of packaging were tested. Acellular porcine tissue matrix samples were inoculated with Bacillus atrophaeus spores. The material was packaged within the TYVEK® pouch and sealed. For some treatment groups, the sealed TYVEK® pouch with the material was then placed in the header pouch at the bottom of the pouch (within the foil area). The header pouches were then sealed at the top of the pouch. For some treatment groups, 15 ml conical tubes were placed in the header pouch prior to sealing to create a wider path for sterilant and CO₂ transmission. Three package configurations were therefore evaluated: 1) TYVEK®-only, 2) TYVEK®-Header, and 3) TYVEK®-Header-Tubes.

Packaged samples were subjected to sterilization treatment, as described above, using sterilant with a 1 hour run time. One run was performed for each package configuration and six units were placed in the chamber for each run. Immediately following each run, the sample was removed from the package system and the Bacillus atrophaeus count was determined by extraction and plating.

Table 3 shows the microbial inactivation of the Bacillus atrophaeus for the three different packaging configurations in terms of log₁₀ reduction. The inactivation using the TYVEK®-Header was the lowest and the most inconsistent. Inactivation using the TYVEK®-only showed the most consistent and highest level of inactivation although the TYVEK®-Header-Tubes was similar to TYVEK®-only. ANOVA and Turkey's multiple comparison analysis was perform for inactivation using the three package configurations. The ANOVA showed a statistical significance difference (P=0.000) for the three groups. The Turkeys test with a family error rate of 5% showed that the TYVEK®-Header had statistically lower inactivation than either TYVEK®-only or TYVEK®-Header-Tubes, but no statistical difference was found between TYVEK®-only and TYVEK®-Header-Tube samples. Therefore, a configuration that provides an open passage to facilitate sterilization with SC—CO₂-PAA is better than a configuration in which the sample is placed in a sealed header pouch.

TABLE 3

Log₁₀ Reduction

| | | TYVEK ® in | TYVEK ® in |
|---|---|---|---|
| | TYVEK ® only | Header pouch | header with tubes |
| | >6.3 | 1.7 | >6.2 |
| | >6.3 | 1.1 | >6.2 |
| | >6.3 | 2.6 | >6.2 |
| | >6.3 | 5.6 | 4.5 |
| | >6.3 | 3.5 | >6.2 |
| | >6.3 | 2.7 | 6.2 |
| mean | >6.3 | 2.9 | 5.9 |
| stdev | N/A | 1.6 | 0.7 |

The microbial inactivation for tissue inoculated with Bacillus atrophaeus contained within the header pouch was lower when the pouch was not held open during treatment. These data indicate that microbial inactivation for a given treatment time can be reduced if the path of the sterilant is constrained.

### Example 3: Inactivation of Microorganisms

Suspensions of microorganisms were prepared using various bacteria (Enterobacter aerogenes, Staphylococcus cohnii, Staphylococcus haemolyticus, Bacillus atrophaeus), yeast (Debaryomyces hansenii), and mold (Penicillium, Aspergillus, Verticillium). The mold and Bacillus atrophaeus suspensions were used to directly inoculate pieces of porcine acellular dermal matrix cut to 5 cm×8 cm and 1 mm thick. The pieces were first blotted until the surfaces appeared dry to remove surface fluid before inoculation. After inoculation, the tissues were rehydrated with a product preservation solution. The microorganisms of the remaining suspensions were used to indirectly inoculate additional pieces of porcine tissue by co-culturing the suspension with the tissue.

After inoculation, all tissue pieces were packaged in TYVEK® pouches and treated in the SC—CO₂ chamber with PAA sterilant for a run time of either 1 or 5 minutes. Pouches are as shown in FIG. 1, wherein after tissue sample placement in the TYVEK® pouch, the TYVEK® pouch was surrounded by mesh and then placed in a foil packaging featuring a TYVEK® header. The foil package featuring the TYVEK® header was sealed along the top. The purpose of the mesh is to hold the TYVEK® header portion of the header pouch open to allow for efficient transport of the sterilant to the product. Bacillus atrophaeus, which was determined to be most resistant to SC—CO₂-PAA sterilization in the initial study, was further studied by varying the treatment running time from 1 to 30 minutes. The SC—CO₂ chamber had the following settings during treatment: temperature 35-41° C., pressure 1365-1455 psi, stirrer speed 650-710 rpm. The final concentration of PAA in the SC—CO₂ chamber was approximately 55 ppm.

After inoculation, the microorganisms were collected by sonicating tissue in extraction fluid, which was then diluted and filtered onto membranes. The membranes were incubated on TSA plates and colony forming units (CFU) were counted. Tissue samples inoculated with microorganisms but not treated with SC—CO₂-FAA were used as controls.

Tables 4 and 5 show microbial inactivation at 1 minute and 5 minute sterilization run times, respectively. Substantial reduction in bacterial CFUs was observed for all organisms except for Bacillus atrophaeus at either run time. One minute sterilization run times resulted in mean log₁₀ reductions of 7 and 8.4 for Enterobacter aerogenes and Debaryomyces hansenii, respectively, while mean log₁₀ reductions greater than 10.1 were observed for both Staphylococcus cohnii and Staphylococcus haemolyticus. Five minute sterilization run times resulted in mean log₁₀ reductions of 5.9, 6.1, and 5.7 for Penicillium, Aspergillus, and Verticillium, respectively. Bacillus atrophaeus observed only a 2.9 and 3.7 mean log₁₀ reduction in CFU with 1 minute and 5 minute sterilizations, respectively.

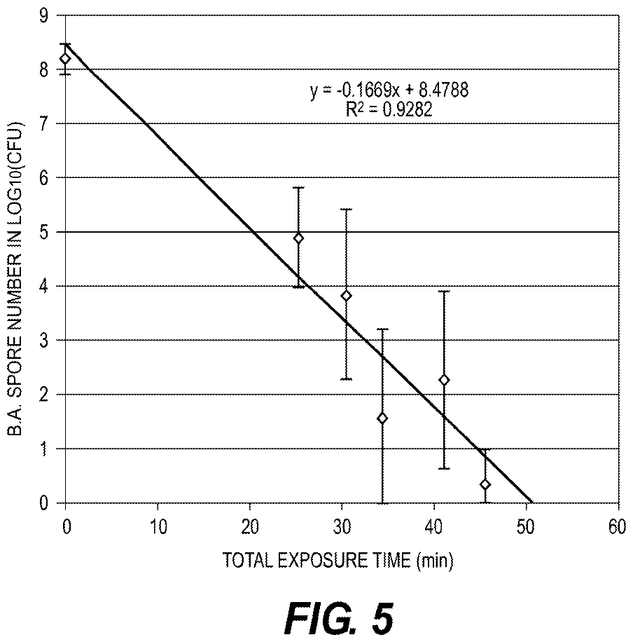

The results of the further study of Bacillus atrophaeus, are shown in Table 6. As demonstrated, CFU dropped to 0 after a 30 minute sterilization run time. As FIG. 5 shows, a strong linear inactivation profile for the spores was observed using total treatment time. This indicated that inactivation could correlate predictably with D₁₀ values and times required for SAL 10⁻⁶ sterilization. The D₁₀ value, the time required to achieve a 90% reduction in active Bacillus atrophaeus spores, was determined to be 6.3 minutes based on the kill rate from the survivor curve. The bioburden was determined by performing the extraction and plating method on tissue that was not inoculated with microorganisms. The mean bioburden (B) for the matrix used was measured to be less than 100 CFU. Therefore, the total exposure time to achieve SAL of 10⁻⁶ was determined to be 52 minutes, using the formula: t≤D₁₀[6+log₁₀(100+B)], where t=exposure time for routine sterilization processing to achieve SAL 10⁻⁶; D₁₀=time to achieve 90% inactivation for the most resistant organism; B=product bioburden.

The average combined time for fill and empty stages was about 25 minutes. The run time to achieve SAL of 10⁻⁶ was therefore, determined to be 27 minutes. A reduction of the viable spores below the detection level after 30 minute sterilization run time using SC—CO₂-PAA confirmed that the minimal run time determined could achieve the industrial sterilization level of SAL 10⁻⁶ with acellular dermal matrix.

TABLE 4
Inactivation of Various Microorganisms by SC-
CO₂-PAA for 1 Minute Sterilization Run Time

| | Log₁₀ CFU | Mean Log₁₀ CFU | Mean Log₁₀ |
|---|---|---|---|
| Organism | (Pretreatment) | (Post-treatment) | Reduction |
| Enterobacter. | 10.2 | 3.3 | 7 |
| aerogenes | | | |
| Staphylococcus, | 10.1 | N/A | >10.1 |
| cohnii | | | |
| Staphylococcus, | 10.1 | N/A | >10.1 |
| haemolyticus | | | |
| Debaryomyces | 9.1 | 0.7 | 8.4 |
| hansenii | | | |
| Bacillus atrophaeus | 8.3 | 5.4 | 2.9 |

TABLE 5
Inactivation of Various Microorganisms by SC-
CO2-PAA for 5 Minute Sterilization Run Time

| | Log₁₀ CFU | Mean Log₁₀ CFU | Mean Log₁₀ |
|---|---|---|---|
| Organism | (Pretreatment) | (Post-treatment) | Reduction |
| Penicillium | 6.3 | 0.4 ± 0.8 | 5.9 ± 0.8 |
| Aspergillus | 6.8 | 0.7 ± 0.8 | 6.1 ± 0.8 |
| Verticillium | 6.7 | 1.0 ± 1.3 | 5.7 ± 1.3 |
| Bacillus atrophaeus | 8.3 | 4.6 ± 1.1 | 3.7 ± 1.1 |

TABLE 6
Inactivation of Bacillus atrophaeus Spores
by SC-CO2-PAA With Different Treatment Times

| Run Time | Total Exposure Time | Average Spore Count |
|---|---|---|
| (min) | (min) | (CFU) |
| 0 | 0 | 2.2 × 10⁸ |
| (Control) | | |
| 1 | 25.8 | 2.7 × 10⁵ |
| 5 | 30.8 | 3.7 × 10⁴ |
| 10 | 34.7 | 5.6 × 10³ |
| 15 | 41.3 | 7.0 × 10³ |
| 20 | 45.8 | 12 |
| 30 | 56.0 | 0 |

Based on these results, the disclosed methods and packaging systems are effective for reduction in the bioburden caused by a variety of microorganisms, including bacteria, yeast, and mold. Further, the disclosed methods and packaging systems are effective in reducing or eliminating microorganisms known to be highly resistant to chemical sterilization, such as Bacillus atrophaeus.

### Example 4: Inactivation of Viruses

Suspensions of porcine encephalomyocarditis virus (EMC), porcine parvovirus (PPV), porcine pseudorabies virus (PRV) and murine leukemia retrovirus (LRV) were prepared in MEM and used to inoculate pieces of porcine acellular matrix cut 2 cm×3 cm, weighing approximately 1 g, at a ratio of 0.5 ml/g. Prior to inoculation, the pieces were blotted to remove surface fluid. After inoculation, 0.1% PAA was added to the tissue at a ratio of 5 ml/g. The pieces were then agitated in the PAA solution, homogenized, and recombined with PAA diluted with PBS to extract the viruses. The extract solution was then used to prepare serial dilutions and plaque forming units (PFU) were quantified using a modified plaque assay. Tissue samples inoculated with virus but not treated with PAA were used as controls.

As Table 7 shows, EMC virus proved more resistant to PAA sterilization alone than the other viruses. EMC virus remained after a 1 or 2 hour treatment with PAA, whereas the other viruses were reduced to below detectable levels at both time points.

TABLE 7
Inactivation of Viruses in PAA Solution for Different
Treatment Times

| | | Treatment Time = 1 h | | Treatment Time = 2 h | |
|---|---|---|---|---|---|
| | Control | Treated | Reduction | Treated | Reduction |
| Virus | (Log₁₀ PFU) | (Log₁₀ PFU) | (Log₁₀ PFU) | (Log₁₀ PFU) | (Log₁₀ PFU) |
| EMC | 7.87 + 0.03 | 4.49 ± 0.09 | 3.38 ± 0.09 | 4.11 ± 0.23 | 3.76 ± 0.23 |
| PPV | 7.73 ± 0.28 | <3.56 | >4.17 | <2.28 | >5.45 |
| PRV | 8.01 ± 0.13 | <2.10 | >5.91 | <0.95 | >7.06 |
| LRV | 6.81 ± 0.26 | <2.94 | >3.87 | <1.67 | >5.14 |

Further study with EMC virus was conducted using SC—CO₂-PAA sterilization. 5 mg pieces of porcine acellular matrix were inoculated with 2.5 ml of virus resuspended in MEM to a viral concentration of about 7×10⁷ PFU per ml. The pieces were then packaged in TYVEK® pouches and subjected to 15 or 30 minute run times of SC—CO₂-PAA treatment. Pouches are as shown in FIG. 1, wherein after sample placement, the TYVEK® pouch was then placed in a foil packaging which was held open during sterilization by a mesh. Following treatment, the tissue was removed from the package, homogenized in DPBS, centrifuged, and filtered. Surviving viruses were determined using the modified plaque assay. One sample that was not treated was used as a control for each time point. SC—CO₂-PAA settings are described in Example 3.

As noted above, EMC virus proved more resistant to PAA sterilization alone than the other viruses. SC—CO₂-PAA treatment however, proved to be effective at inactivating EMC virus at both the 30 minute and 15 minute run times. After a 15 minute run time using SC—CO₂-PAA, samples inoculated with virus at 7.77±0.08 log₁₀ observed a reduction in virus levels of more than 6.44 log₁₀ and no surviving virus was detected.

The results of the virus inactivation study demonstrate that the disclosed methods and packaging systems are effective in conjunction with a variety of viruses. The results also demonstrate that the disclosed methods and packaging systems are effective in conjunction with viruses known to be highly resistant to PAA treatment alone, such as EMC virus.

### Example 5: Effect of Sterilization on Acellular Dermal Matrix

Porcine acellular matrix was packaged in a TYVEK® pouch and subjected to SC—CO₂-PAA sterilization with a 1.5 hour run time. SC—CO₂-PAA settings are described in Example 3. After sterilization, the effects of treatment on the biochemical properties of the matrix were evaluated using enzyme digestion analysis. Physical properties of the matrix after treatment were evaluated using mechanical testing.

For the enzyme digestion analysis, tissue matrix samples of about 70 mg were digested in 60 ul Tris-HCl buffer, pH 7.5, containing 2500 U/mL collagenase at 37° C. for 6 hours with agitation. The samples were then centrifuged and decanted, and the remaining solid was freeze-dried and weighed. The percentage of each sample by weight remaining after digestion was calculated. Any increase in the susceptibility to digestion as a result of treatment would be undesirable.

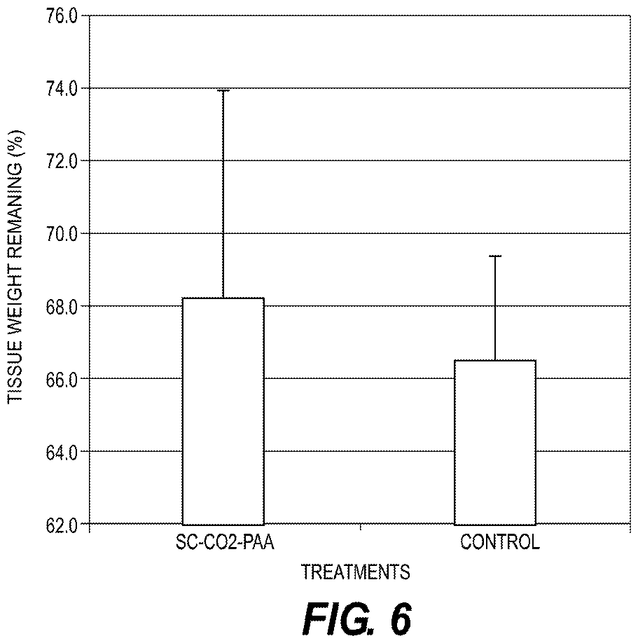

As shown in FIG. 6, collagenase digestion analysis showed that there was no statistically determinable difference in susceptibility to digestion after treatment with SC—CO₂-PAA.

Tensile and tear strengths of treated samples were measured and compared to untreated control samples using an Instron system. A crosshead speed of 1.65 cm min⁻¹ was used for both studies. Maximum load, stress, and elasticity were determined when evaluating tensile strength. When testing tear strength, each test sample was cut to 8 cm×2 cm with a 3 cm slit at the center of the width. Results are shown in Table 8. In comparison to the controls, the SC—CO₂-PAA treated samples showed comparable maximum load, maximum stress, elasticity, and tear strength. Only the elasticity of the treated group appeared significantly lower than the control group.

TABLE 8
Effect of SC-COVPAA on Tissue Tensile and Tear Strength

| Sample ID | SC-CO₂-PAA | Control |
|---|---|---|
| Max load (N cm−1) | 320 ± 62 | 289 ± 61 |
| Max Stress (MPa) | 20.6 ± 3.0 | 18.3 ± 2.8 |
| Elasticity (N cm−1) | 897 ± 116 | 1089 ± 237 |
| Tear (N cm−1) | 30.7 ± 9.2 | 31.0 ± 7.6 |

Thus, the results demonstrate that the disclosed methods and packaging systems do not adversely impact the biochemical or physical properties of acellular tissue. Maintenance of tensile and tear strength is important since acellular tissues can be used in implantation procedures to help repair, reinforce, or augment patient tissue.

What is claimed is:

1. A packaging system for a medical device, comprising an outer package, the outer package comprising a first portion and a second portion, wherein the first portion is permeable to supercritical carbon dioxide (SC—CO₂) and a sterilant and impermeable to bacteria, and wherein when the second portion is sealed it is impermeable to moisture;
an inner structure configured to hold the second portion open during sterilization, wherein the inner structure is separate from the outer package; and
an inner package that is permeable to SC—CO₂ and a sterilant, wherein the inner package is positioned within the inner structure.

2. The packaging system of claim 1, wherein the first portion comprises flash spun high-density polyethylene fibers.

3. The packaging system of claim 1, wherein the first portion comprises medical grade paper.

4. The packaging system of claim 1, wherein the second portion comprises foil.

5. The packaging system of claim 1, wherein the inner package comprises flash spun high-density polyethylene fibers.

6. The packaging system of claim 1, wherein the inner structure is permeable to supercritical carbon dioxide SC—CO₂ and the sterilant.

7. A method of sterilization, comprising:
selecting a medical device for sterilization;
placing the medical device in an inner package that is permeable to SC—CO₂ and a sterilant;
placing the inner package and the medical device inside an inner structure, wherein the inner structure is impermeable to SC—CO₂ and the sterilant;
placing the inner structure, the inner package, and the medical device in a first outer package, wherein the first outer package comprises a first portion that is permeable to SC—CO₂ and the sterilant and is impermeable to bacteria and a second portion that is impermeable to moisture when sealed; and
treating the medical device with SC—CO₂ and the sterilant, wherein the inner structure is configured to hold the second portion open during treatment.

8. The method of claim 7, wherein the medical device comprises acellular tissue matrix.

9. The method of claim 7, wherein the medical device comprises demineralized bone matrix.

10. The method of claim 7, wherein the sterilant comprises peracetic acid (PAA).

11. The method of claim 10, wherein the sterilant comprises a peroxide.

12. The method of claim 11, wherein the peroxide is H₂O₂.

13. The method of claim 12, wherein the concentrations of PAA and H₂O₂ in the sterilant are 10-14% and 1-3%, respectively.

14. The method of claim 7, wherein during the treating step, pressure and temperature are kept constant.

15. The method of claim 7, further comprising sealing the second portion subsequent to sterilization.

16. The method of claim 7, wherein the first portion comprises flash spun high density polyethylene.

17. The method of claim 7, wherein the second portion comprises foil.

18. The method of claim 7, wherein placing the inner structure inside the outer package maintains a flow path within the outer package during sterilization.

19. The method of claim 7, wherein the structure maintains an opening within the second portion of the outer package during sterilization of the medical device.

20. The method of claim 7, further comprising sealing the first portion prior to sterilization.
